(19) United States
(12) Patent Application Publication
(10) Pub. No.: US 2026/0218069 A1
(43) Pub. Date: Jul. 30, 2026

(54) HYDROCARBON TREATMENT INCLUDING PYROLYSIS OIL EXTRACTION

(71) Applicant: NOVA CHEMICALS (INTERNATIONAL) S.A., Fribourg (CH)

(72) Inventors: Bolaji OLAYIWOLA, Calgary (CA); Yoonhee KIM, Calgary (CA)

(73) Assignee: NOVA CHEMICALS (INTERNATIONAL) S.A., Fribourg (CH)

(21) Appl. No.: 19/142,483

(22) PCT Filed: Jan. 2, 2024
(86) PCT No.: PCT/IB2024/050031

§ 371 Date: Jun. 23, 2025

Related U.S. Application Data
(60) Provisional application No. 63/478,816, filed on Jan. 6, 2023.

(51) Int. Cl. C10G 69/06

(52) U.S. Cl. CPC C10G 69/06; C10G 2300/1092; C10G 2300/202; C10G 2300/205; C10G 2300/802; C10G 2300/807; C10G 2400/20

(57) ABSTRACT

The present disclosure relates to hydrocarbon treatments including oxidative dehydrogenation of ethane-containing feed streams and extraction of contaminants from pyrolysis oil.

### TECHNICAL FIELD

The present disclosure relates to hydrocarbon treatments including oxidative dehydrogenation of ethane-containing feed streams and extraction of contaminants from pyrolysis oil.

### BACKGROUND ART

Pyrolysis of plastic waste yields a liquid product, referred to as pyrolysis oil, that can be cracked to form light olefins such as ethylene. However, contaminants present in pyrolysis oils can make them unsuitable as a direct feedstock for steam cracking processes. For example, heteroatom- and metal-containing contaminants can cause corrosion, catalyst poisoning, increased coking, and/or increased fouling in a cracking reactor.

Conventionally, cracking feedstock contaminants are removed by hydrotreatment, a process in which contaminants are reacted with hydrogen at high temperature and pressure in the presence of a catalyst. Hydrotreatment is also exothermic, and thus must be carefully monitored and controlled to avoid thermal runaway. Accordingly, there is a need for improved methods for removing contaminants from cracking feedstocks such as pyrolysis oils.

### SUMMARY OF INVENTION

Provided in the present disclosure is a method of treating hydrocarbons, the method including contacting an oxidative dehydrogenation feed stream including ethane and oxygen with an oxidative dehydrogenation catalyst to form a dehydrogenated stream including ethylene, water, and acetic acid; separating at least a portion of the dehydrogenated stream to form a gaseous product stream including ethylene and a byproduct stream including water and acetic acid; contacting a pyrolysis oil including a contaminant with at least a portion of the byproduct stream including water and acetic acid to form a mixture including the pyrolysis oil, water, and acetic acid; and separating the mixture to form a treated stream including a portion of the pyrolysis oil and a waste stream including water and at least a portion of the contaminant or a derivative thereof.

In some embodiments, the dehydrogenation feed stream further includes a diluent. In some embodiments, the diluent includes steam, carbon dioxide, or both. In some embodiments, the dehydrogenated stream further includes ethane, carbon dioxide, carbon monoxide, or any combination thereof. In some embodiments, the product stream further includes ethane, carbon dioxide, carbon monoxide, or any combination thereof.

In some embodiments, the contaminant includes arsenic, calcium, iron, lead, aluminum, mercury, zinc, potassium, silica, sodium, copper, nickel, vanadium, sulfur, phosphorus, chlorine, a fluoride, or any combination thereof. In some embodiments, the derivative of the contaminant includes an acetate salt. In some embodiments, the treated stream includes about 0 ppbw to less than about 5 ppbw arsenic, calculated on an elemental basis. In some embodiments, the treated stream includes about 0 ppbw to less than about 500 ppbw of calcium, calculated on an elemental basis. In some embodiments, the treated stream includes about 0 ppbw to less than about 8 ppbw of chromium, calculated on an elemental basis. In some embodiments, the treated stream includes about 0 ppbw to less than about 150 ppbw of iron, calculated on an elemental basis. In some embodiments, the treated stream includes about 0 ppbw to less than about 50 ppbw of lead, calculated on an elemental basis. In some embodiments, the treated stream includes about 0 ppbw to less than about 1 ppbw of aluminum, calculated on an elemental basis. In some embodiments, the treated stream includes about 0 ppbw to less than about 5 ppbw of mercury, calculated on an elemental basis. In some embodiments, the treated stream includes about 0 ppbw to less than about 1 ppbw of zinc, calculated on an elemental basis. In some embodiments, the treated stream includes about 0 ppbw to less than about 35,000 ppbw of potassium, calculated on an elemental basis. In some embodiments, the treated stream includes about 0 ppbw to less than about 1,000 ppbw of silicon, calculated on an elemental basis.

In some embodiments, the treated stream includes about 0 ppbw to less than about 300 ppbw of sodium, calculated on an elemental basis. In some embodiments, the treated stream includes about 0 ppbw to less than about 50,000 ppbw of copper, calculated on an elemental basis. In some embodiments, the treated stream includes about 0 ppbw to less than about 100,000 ppbw of nickel, calculated on an elemental basis. In some embodiments, the treated stream includes about 0 ppbw to less than about 50 ppbw of vanadium, calculated on an elemental basis. In some embodiments, the treated stream includes about 0 ppbw to less than about 500,000 ppbw of sulfur, calculated on an elemental basis. In some embodiments, the treated stream includes about 0 ppbw to less than about 900 ppbw of phosphorus, calculated on an elemental basis. In some embodiments, the treated stream includes about 0 ppbw to less than about 3,000 ppbw of chlorine, calculated on an elemental basis. In some embodiments, the treated stream includes about 0 ppbw to less than about 2,000 ppbw of fluorine, calculated on an elemental basis.

In some embodiments, the byproduct stream includes about 0.1 wt % to about 30 wt % of acetic acid. In some embodiments, the byproduct stream includes about 1 wt % to about 30 wt % of acetic acid. In some embodiments, the mixture includes an emulsion of the pyrolysis oil and the byproduct stream.

In some embodiments, a weight ratio of the pyrolysis oil to the byproduct stream present in the mixture is about 5:1 to about 1:5. In some embodiments, a weight ratio of the pyrolysis oil to the byproduct stream present in the mixture is about 2:1 to about 1:2.

In some embodiments, the waste stream further includes acetic acid. In some embodiments, the treated stream further includes acetic acid.

In some embodiments, the method further includes contacting at least a portion of the treated stream including pyrolysis oil and acetic acid with water to form a mixture including the pyrolysis oil, water, and acetic acid; and separating the mixture to form a washed stream including at least a portion of the pyrolysis oil and a second waste stream including water and at least a portion of the acetic acid from the treated stream.

In some embodiments, the method further includes pyrolyzing a plastic to form at least a portion of the pyrolysis oil including the contaminant. In some embodiments, the method further includes contacting a hydrocracking feed stream including at least a portion of the treated stream including pyrolysis oil and hydrogen with a hydrocracking catalyst to form a hydrocracked stream including ethane. In some embodiments, the method further includes heating a steam cracking feed stream including water and at least a portion of the hydrocracked stream including ethane to form a cracked stream including ethylene.

Also provided in the present disclosure is a system for treating hydrocarbons, the system including an oxidative dehydrogenation reactor configured to contact an oxidative dehydrogenation feed stream including ethane and oxygen with an oxidative dehydrogenation catalyst to form a dehydrogenated stream including ethylene, water, and acetic acid; a liquid-gas separator configured to separate at least a portion of the dehydrogenated stream to form a gaseous product stream including ethylene and a byproduct stream including water and acetic acid; and a liquid-liquid extractor configured to contact a pyrolysis oil including a contaminant with at least a portion of the byproduct stream including water and acetic acid to form a mixture including the pyrolysis oil, water, and acetic acid, and separate the mixture to form a treated stream including a portion of the pyrolysis oil and a waste stream including water and at least a portion of the contaminant or a derivative thereof.

In some embodiments, the liquid-liquid extractor includes an extraction column configured to form the mixture and separate the mixture. In some embodiments, the liquid-liquid extractor includes a static mixer configured to form the mixture and a liquid-liquid separator configured to separate the mixture.

In some embodiments, the system further includes a second liquid-liquid extractor configured to contact at least a portion of the treated stream including pyrolysis oil and acetic acid to form a mixture including the pyrolysis oil, water, and acetic acid, and separate the mixture to form a washed stream including at least a portion of the pyrolysis oil and a second waste stream including water and at least a portion of the acetic acid from the treated stream.

In some embodiments, the system further includes a hydrocracking reactor configured to contact a hydrocracking feed stream including at least a portion of the treated stream including pyrolysis oil and hydrogen with a hydrocracking catalyst to form a hydrocracked stream including ethane. In some embodiments, the system further includes a hydrocracking reactor configured to heat a steam cracking feed stream including water and at least a portion of the hydrocracked stream including ethane to form a cracked stream including ethylene.

### BRIEF DESCRIPTION OF DRAWINGS

FIG. 1 is a schematic diagram of a system for treating hydrocarbons.

FIG. 2 is a process flow diagram of a method for treating hydrocarbons.

FIG. 3 is a process flow diagram of a method for treating hydrocarbons.

### DESCRIPTION OF EMBODIMENTS

The present disclosure relates to methods and systems for extracting contaminants from pyrolysis oil using an acetic acid-containing byproduct of an integrated oxidative dehydrogenation process. The operational methods and systems described herein provide a process for reducing the levels of one or more pyrolysis oil contaminants. In some embodiments, the operational methods and systems provide a process for reducing the levels of one or more pyrolysis oil contaminants to be within industrial limits for cracking processes such as steam cracking. In some embodiments, the methods and systems require less energy as compared to those including hydrotreatment. In some embodiments, the methods and systems involve simpler equipment and/or milder conditions as compared to those including hydrotreatment.

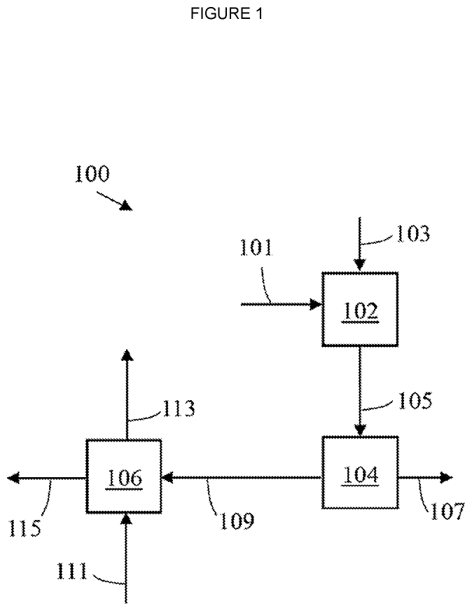

FIG. 1 is a schematic illustration of a system 100 for treating hydrocarbons in accordance with certain embodiments of the present disclosure. The system includes an oxidative dehydrogenation reactor 102, a liquid-gas separator 104, and a liquid-liquid extractor 106.

Liquid and gaseous streams and materials used in the system represented in FIG. 1 can be directed and transferred through suitable transfer lines, conduits, and piping. Particular elements may be physically juxtaposed and, where appropriate, may have flexible regions, rigid regions, or a combination of both. In directing streams of compounds, intervening apparatuses and/or optional treatments may be included. For example, pumps, valves, manifolds, gas and liquid flow meters and distributors, sampling and sensing devices, and other equipment (e.g., for monitoring, controlling, adjusting, and/or diverting pressures, flows and other operating parameters) can be included in the system.

An oxidant stream 101 including oxygen (O₂) and an oxidative dehydrogenation feed stream 103 including ethane (C₂H₆) are directed to the oxidative dehydrogenation reactor 102 including an oxidative dehydrogenation catalyst (not shown). Suitable configurations of the oxidative dehydrogenation reactor are known in the art. For example, the oxidative dehydrogenation reactor can include one shell-and-tube reactor, or two or more shell-and-tube reactors (e.g., in series and/or in parallel). In some embodiments, the oxidant stream, the oxidative dehydration feed stream, or both include a diluent. In some examples, the diluent can help to control the heat of reaction released. In some embodiments, the diluent can help to maintain the temperature of the reactor outside the flammable zone for the hydrocarbon-oxidant mixture present therein. Certain diluents can be reactive, and can change the selectivity of ethylene. The diluent can include, for example, steam (H₂O), carbon dioxide (CO₂), nitrogen (N₂), helium (He), argon (Ar), and the like. In some embodiments, the diluent includes steam, carbon dioxide, or both.

In some embodiments, the composition of the oxidant stream and the oxidative dehydration feed stream can be selected to provide to the reactor, in combination, a mixture including about 1 wt % to about 30 wt % oxygen, e.g., about 1 wt % to about 25 wt %, about 2 wt % to about 20 wt %, or about 5 wt % to about 15 wt % oxygen. In some embodiments, the composition of the oxidant stream and the oxidative dehydration feed stream can be selected to provide to the reactor, in combination, a mixture including about 1 wt % to about 40 wt % of one or more C₂-C₄ alkanes, e.g., about 1 wt % to about 35 wt %, about 2.5 wt % to about 30 wt %, or about 5 wt % to about 25 wt % of one or more C₂-C₄ alkanes. For example, the composition of the oxidant stream and the oxidative dehydration feed stream can be selected to provide to the reactor, in combination, a mixture including about 5 wt % to about 15 wt % oxygen, and about 5 wt % to about 25 wt % ethane. In certain such embodiments, the mixture further includes steam, carbon dioxide, or both. In some embodiments, the oxidative dehydrogenation feed stream includes the product of a hydrocracking process. For example, in some embodiments, the oxidative dehydrogenation feed stream includes the product of hydrocracking a pyrolysis oil, e.g., derived from pyrolysis of a plastic (e.g., a waste plastic) as described herein. Other suitable sources of ethane-containing feed streams are known in the art.

Non-limiting examples of the oxidative dehydrogenation catalyst include those containing one or more mixed metal oxides. In some embodiments, the catalyst is selected from:
i) catalysts of the formula:

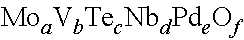

where a, b, c, d, e and f are the relative atomic amounts of the elements Mo, V, Te, Nb, Pd and O, respectively; and where a=1, b=0.01 to 1.0, c=0.01 to 1.0, d=0.01 to 1.0, 0.00≤e≤0.10 and f is a number to at least satisfy the valence state of the metals in the catalyst;
ii) catalysts of the formula:

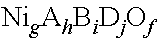

where g is a number from 0.1 to 0.9, such as from 0.3 to 0.9, from 0.5 to 0.85, or from 0.6 to 0.8; h is a number from 0.04 to 0.9; i is a number from 0 to 0.5; j is a number from 0 to 0.5; and f is a number to at least satisfy the valence state of the catalyst; A is chosen from Ti, Ta, V, Nb, Hf, W, Y, Zn, Zr, Si and Al or mixtures thereof, B is chosen from La, Ce, Pr, Nd, Sm, Sb, Sn, Bi, Pb, Tl, In, Te, Cr, Mn, Mo, Fe, Co, Cu, Ru, Rh, Pd, Pt, Ag, Cd, Os, Ir, Au, Hg, and mixtures thereof, D is chosen from Ca, K, Mg, Li, Na, Sr, Ba, Cs, and Rb and mixtures thereof; and O is oxygen;
iii) catalysts of the formula:

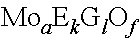

where E is chosen from Ba, Ca, Cr, Mn, Nb, Ta, Ti, Te, V, W and mixtures thereof, G is chosen from Bi, Ce, Co, Cu, Fe, K, Mg, V, Ni, P, Pb, Sb, Si, Sn, Ti, U, and mixtures thereof, a=1; k is 0 to 2; 1=0 to 2, with the proviso that the total value of 1 for Co, Ni, Fe and mixtures thereof is less than 0.5; and f is a number to at least satisfy the valence state of the metals in the catalyst;
iv) catalysts of the formula:

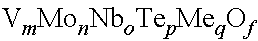

where Me is chosen from Ta, Ti, W, Hf, Zr, Sb and mixtures thereof, m is from 0.1 to 3; n is from 0.5 to 1.5; o is from 0.001 to 3; p is from 0.001 to 5; q is from 0 to 2; and f is a number to at least satisfy the valence state of the metals in the catalyst; and
v) catalysts of the formula:

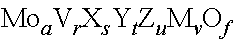

where X is at least one of Nb and Ta; Y is at least one of Sb and Ni; Z is at least one of Te, Ga, Pd, W, Bi and Al; M is at least one of Fe, Co, Cu, Cr, Ti, Ce, Zr, Mn, Pb, Mg, Sn, Pt, Si, La, K, Ag and In; a=1.0 (normalized); r=0.05 to 1.0; s=0.001 to 1.0; t=0.001 to 1.0; u=0.001 to 0.5; v=0.001 to 0.3; and f is a number to at least satisfy the valence state of the metals in the catalyst.
vi) a mixed metal oxide having the empirical formula:

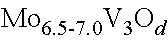

where d is a number to at least satisfy the valence of the metals in the catalyst; and
vii) a mixed metal oxide having the empirical formula:

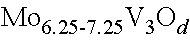

where d is a number to at least satisfy the valence of the metals in the catalyst.

In some embodiments, the catalyst is supported on or agglomerated with a binder. Some binders include acidic, basic, or neutral binder slurries of TiO₂, ZrO₂Al₂O₃, AlO(OH) and mixtures thereof. Another useful binder includes Nb₂O₅. The agglomerated catalyst can be extruded in a suitable shape, such as rings, spheres, or saddles, among others, of a size typically used in fixed bed reactors. When the catalyst is extruded, various extrusion aids known in the art can be used. In some cases, the resulting support may have a cumulative surface area of less than 35 m²/g as measured by BET, in some cases, less than 20 m²/g, in other cases, less than 3 m²/g. and a cumulative pore volume from 0.05 to 0.50 cm³/g.

The oxidative dehydrogenation feed stream and oxidant stream can be contacted with the oxidative dehydrogenation catalyst under any condition suitable for the oxidative dehydrogenation reaction. For example, in some embodiments, the feed stream, oxidant stream, and catalyst are contacted at a temperature of between about 300° C. and about 500° C., or between about 300° C. and about 450° C., or between about 330° C. and about 425° C. In various embodiments, the feed stream, oxidant stream, and catalyst are contacted at a pressure of between about 0.5 psig and about 100 psig (about 3.447 kPag and about 689.47 kPag), or between about 15 psig and about 50 psig (about 103.4 kPag and about 344.73 kPag). In various embodiments, the residence time of the one or more alkanes in the oxidative dehydrogenation reactor is between about 0.002 seconds and about 30 seconds, or between about 1 seconds and about 10 seconds. In various embodiments, the gas hourly space velocity (GHSV) of the oxidative dehydrogenation reactor is between about 400 h⁻¹ and about 30000 h⁻¹, or greater than about 1000 h⁻¹. In various embodiments, the weight hourly space velocity (WHSV) is between about 0.4 h⁻¹ and about 30 h⁻¹. In some embodiments, the gas velocity is between about 5 cm/sec and about 500 cm/sec.

Ethane from the feed stream 103 is dehydrogenated in the oxidative dehydrogenation reactor 102 to form a dehydrogenated stream 105 including ethylene (C₂H₄) and water. In some embodiments, the selectivity of the process for ethylene is greater than about 85%, or greater than about 90%, or greater than about 95%, or greater than about 98%. In some embodiments, the space-time yield of ethylene (productivity) in g/hour per kg of the catalyst is at least about 50, or at least about 1500, or at least about 3000, or at least about 3500, at a temperature of the oxidative dehydrogenation reactor of between about 330° C. to 500° C. In some embodiments, the dehydrogenated stream further includes ethane, carbon dioxide (CO₂), carbon monoxide (CO), acetic acid, or any combination thereof.

The dehydrogenated stream 105 is separated in liquid-gas separator 104 to form a gaseous product stream 107 including ethylene and a liquid byproduct stream 109 including water and acetic acid. Separators suitable for gas-liquid separations are known in the art. In some embodiments, the separator includes a condenser (e.g., a shell-and-tube heat exchanger). In some embodiments, the separator includes a flash tank. In some embodiments, the separator includes a scrubber, for example, a quench tower, a spray tower, a venture scrubber, a tray tower, or a packed tower. In certain such embodiments, the byproduct stream includes water fed to the scrubber (e.g., as scrubbing liquid).

In some embodiments, the product stream includes about 60 wt % to about 99 wt % of ethylene, for example, about 60 wt % to about 95 wt %, about 70 wt % to about 99 wt %, about 70 wt % to about 95 wt %, about 80 wt % to about 99 wt %, or about 80 wt % to about 95 wt % of ethylene. In some embodiments, the product stream further includes carbon monoxide, carbon dioxide, ethane, or any combination thereof.

The byproduct stream can include water and acetic acid. In some embodiments, the byproduct stream includes about 0.1 wt % to about 30 wt % of acetic acid. For example, in some embodiments, the byproduct stream includes about 0.5 wt % to about 30 wt %, about 1 wt % to about 30 wt %, about 1 wt % to about 25 wt %, about 1 wt % to about 20 wt %, about 5 wt % to about 30 wt %, about 5 wt % to about 25 wt %, about 5 wt % to about 20 wt %, about 10 wt % to about 30 wt %, about 10 wt % to about 25 wt %, or about 10 wt % to about 20 wt % of acetic acid. In some embodiments, the byproduct stream includes about 10 wt %, about 15 wt %, about 20 wt %, or about 25 wt % of acetic acid. In some examples, the oxidative dehydrogenation and/or liquid-gas separation conditions can be selected to provide a byproduct stream having a particular acetic acid content, e.g., from about 10 wt % to about 25 wt % of acetic acid. For example, operating conditions of the oxidative dehydrogenation reactor (e.g., temperature and/or pressure) can be adjusted to vary the acetic acid content of the byproduct stream (e.g., relative to produced ethylene). In another example, recycling of produced ethylene into the oxidative dehydration reactor can increase the acetic acid content of the byproduct stream. The use of diluents (e.g., steam) in the oxidative dehydrogenation reactor can also affect the acetic acid content of the byproduct stream.

In some examples, the acetic acid content of the byproduct stream can be increased (e.g., before introduction to the liquid-liquid extractor) using separation methods known in the art, such as distillation or liquid-to-liquid extraction.

At least a portion of the byproduct stream 109 including water and acetic acid and a pyrolysis oil stream 111 including a contaminant are directed to the liquid-liquid extractor 106, and contacted to form a mixture including the pyrolysis oil, water, and acetic acid. In some embodiments, the pyrolysis oil includes at least a portion of the pyrolysis product of a plastic (e.g., a waste plastic). In certain such embodiments, the plastic includes a polyester such as polyethylene terephthalate (PET) and polycaprolactone, a polyolefin such as low-density polyethylene (LDPE), linear low density polyethylene (LLDPE) medium-density polyethylene (MDPE), high-density polyethylene (HDPE), and polypropylene (PP), a polyvinyl chloride (PVC), a polystyrene (PS), a polycarbonate, a polylactide, a polyether, a polyacrylate, an acrylonitrile rubber such as acrylonitrile butadiene styrene (ABS), styrene acrylonitrile resin (SAN), acrylonitrile styrene acrylate (ASA), and nitrile rubber (NBR), a fiberglass, a nylon, a polyurethane, any copolymer thereof, or any combination thereof. Suitable methods and systems for pyrolysis are known in the art. For example, in some embodiments, the pyrolysis includes thermal pyrolysis without a catalyst. In some embodiments, the pyrolysis includes catalytic pyrolysis. In some embodiments, the pyrolysis includes hydrothermal pyrolysis. In certain such embodiments, plastic (e.g., a waste plastic) and water are mixed in a reactor at high pressure (e.g., about 10 MP) and mild temperature (e.g., about 300° C.) to form the pyrolysis oil.

In some embodiments, a paraffin content of the pyrolysis oil is about 16 wt % to about 42 wt %, or about 20 wt % to about 26 wt %. In some embodiments, a naphthene content of the pyrolysis oil is about 2 wt % to about 22 wt %, about 14 wt % to about 22 wt %, or about 2 wt % to about 21 wt %. In some embodiments, an olefin content of the pyrolysis oil is about 8 wt % to about 48 wt %, about 8 wt % to about 42 wt %, or about 16 wt % to about 48 wt %. In some embodiments, an aromatics content of the pyrolysis oil is about 3 wt % to about 37 wt %, about 28 wt % to about 39 wt %, or about 3 wt % to about 37 wt %. In some embodiments, a boiling point range of the pyrolysis oil is about 15° C. to about 600° C. In some embodiments, a hydrocarbon range of the pyrolysis oil is C₅ to C₅₅.

In some embodiments, the contaminant includes arsenic, calcium, chromium, iron, lead, aluminum, mercury, oxygen, zinc, potassium, silicon, sodium, copper, nickel, vanadium, sulfur, phosphorus, chlorine, fluorine, or any combination thereof. Such contaminants can be present in the pyrolysis oil in elemental form or in another form, such as an oxide, a salt, an organometallic compound, and the like. In some embodiments, the contaminant includes silica. In some embodiments, the contaminant includes a fluoride salt.

In some embodiments, the mixture includes an emulsion of the pyrolysis oil and the byproduct stream. In some embodiments, the pyrolysis oil and the byproduct stream are present in the mixture in a weight ratio of about 5:1 to about 1:5, about 5:1 to about 1:3, about 5:1 to about 1:2, about 3:1 to about 1:5, about 3:1 to about 1:3, about 3:1 to about 1:2, about 2:1 to about 1:5, about 2:1 to about 1:3, or about 2:1 to about 1:2. In some embodiments, the pyrolysis oil and the byproduct stream are present in the mixture in a weight ratio of about 3:1, about 2:1, about 1.5:1, about 1:1, about 1:1.5, about 1:2, or about 1:3.

In the mixture, contaminants can partition from the pyrolysis oil into the byproduct stream. In some embodiments, contaminants react with acetic acid present in the mixture to form a derivative (e.g., an acetate salt) having an increased solubility in water. Such derivatives can partition into the byproduct stream from the pyrolysis oil. In some embodiments, the derivative of the contaminant includes an acetate salt.

The mixture is then separated to form a treated stream 113 including a portion of the pyrolysis oil and a waste stream 115 including water and at least a portion of the contaminant (e.g., as described herein) or a derivative thereof. In some embodiments, an unreacted portion of acetic acid from the byproduct stream is present in the treated stream, the contaminant stream, or both.

Suitable liquid-liquid extractors are known in the art. For example, in some embodiments, the liquid-liquid extractor includes an extraction column. In certain embodiments, the liquid-liquid extractor includes two or more extraction columns (e.g., in series and/or in parallel). The liquid-liquid extractor can be configured, for example, for single-stage extraction, cross-flow extraction, or countercurrent extraction. The liquid-liquid extractor can, in some examples, include other typical components of an extraction system, such as a raffinate stripping column and/or a solvent recovery column.

In certain such embodiments, the extraction column includes a static extraction column, such as a sieve tray extraction column. In other such embodiments, the extraction column includes an agitated extraction column, such as a rotating impeller extraction column. Liquid-liquid extractors can be configured to form the mixture including the pyrolysis oil, water, and acetic acid, and to separate the mixture to form the treated stream and the waste stream. For example, in certain such embodiments, the liquid-liquid extractor includes a mixer-decanter tank, a mixer-settler apparatus, a centrifugal extractor, or an extraction column such as a packed column, a sieve tray column, a rotating disc contactor (RDC) column, a SCHEIBEL® column, a KARR© reciprocating plate column, and the like.

In another example, in some embodiments, the liquid-liquid extractor includes a static mixer configured to form the mixture, and a liquid-liquid separator configured to separate the mixture. For example, in certain such embodiments, the liquid-liquid separator includes a three-phase separator.

The treated stream can include a reduced amount of contaminants as compared to the pyrolysis oil before contacting the byproduct stream. In some embodiments, one or more contaminants remaining in the treated stream are present in an amount within an industrial limit for cracking feedstock.

In some embodiments, the treated stream includes about 0 ppbw to less than about 5 ppbw arsenic, calculated on an elemental basis.

In some embodiments, the treated stream includes about 0 ppbw to less than about 500 ppbw of calcium, calculated on an elemental basis.

In some embodiments, the treated stream includes about 0 ppbw to less than about 8 ppbw of chromium, calculated on an elemental basis. In some embodiments, the treated stream includes less than about 95%, e.g., less than about 85%, of the chromium present in the pyrolysis oil before contacting the byproduct stream.

In some embodiments, the treated stream includes about 0 ppbw to less than about 150 ppbw of iron, calculated on an elemental basis. In some embodiments, the treated stream includes about 0 ppbw to less than about 1 ppbw of iron, calculated on an elemental basis. In some embodiments, the treated stream includes less than about 70%, e.g., less than about 60%, of the iron present in the pyrolysis oil before contacting the byproduct stream.

In some embodiments, the treated stream includes about 0 ppbw to less than about 50 ppbw of lead, calculated on an elemental basis.

In some embodiments, the treated stream includes about 0 ppbw to less than about 1 ppbw of aluminum, calculated on an elemental basis.

In some embodiments, the treated stream includes about 0 ppbw to less than about 5 ppbw of mercury, calculated on an elemental basis.

In some embodiments, the treated stream includes about 0 ppbw to less than about 1 ppbw of zinc, calculated on an elemental basis.

In some embodiments, the treated stream includes about 0 ppbw to less than about 35,000 ppbw, about 0 ppbw to less than about 10,000 ppbw, about 0 ppbw to less than about 5,000 ppbw, or about 0 ppbw to less than about 1,000 ppbw of potassium, calculated on an elemental basis. In some embodiments, the treated stream includes about 0 ppbw to less than about 500 ppbw of potassium, calculated on an elemental basis. In some embodiments, the treated stream includes less than about 95%, e.g., less than about 85%, of the potassium present in the pyrolysis oil before contacting the byproduct stream.

In some embodiments, the treated stream includes about 0 ppbw to less than about 1,000 ppbw of silicon, calculated on an elemental basis.

In some embodiments, the treated stream includes about 0 ppbw to less than about 300 ppbw of sodium, calculated on an elemental basis. In some embodiments, the treated stream includes about 0 ppbw to less than about 125 ppbw of sodium, calculated on an elemental basis. In some embodiments, the treated stream includes less than about 10%, e.g., less than about 5%, of the sodium present in the pyrolysis oil before contacting the byproduct stream.

In some embodiments, the treated stream includes about 0 ppbw to less than about 50,000 ppbw of copper, calculated on an elemental basis. In some embodiments, the treated stream includes about 0 ppbw to less than about 10,000 ppbw, about 0 ppbw to less than about 1,000 ppbw, about 0 ppbw to less than about 100 ppbw, or about 0 ppbw to less than about 25 ppbw of copper, calculated on an elemental basis. In some embodiments, the treated stream includes less than about 20%, e.g., less than about 10%, of the copper present in the pyrolysis oil before contacting the byproduct stream.

In some embodiments, the treated stream includes about 0 ppbw to less than about 100,000 ppbw of nickel, calculated on an elemental basis. In some embodiments, the treated stream includes about 0 ppbw to less than about 50,000 ppbw, about 0 ppbw to less than about 10,000 ppbw, about 0 ppbw to less than about 1,000 ppbw, about 0 ppbw to less than about 100 ppbw, or about 0 ppbw to less than about 20 ppbw of nickel, calculated on an elemental basis. In some embodiments, the treated stream includes less than about 85%, e.g., less than about 75%, of the nickel present in the pyrolysis oil before contacting the byproduct stream.

In some embodiments, the treated stream includes about 0 ppbw to less than about 50 ppbw of vanadium, calculated on an elemental basis.

In some embodiments, the treated stream includes about 0 ppbw to less than about 500,000 ppbw of sulfur, calculated on an elemental basis. In some embodiments, the treated stream includes about 0 ppbw to less than about 100,000 ppbw, about 0 ppbw to less than about 50,000 ppbw, or about 0 ppbw to less than about 15,000 ppbw of sulfur, calculated on an elemental basis. In some embodiments, the treated stream includes less than about 75%, e.g., less than about 60%, of the sulfur present in the pyrolysis oil before contacting the byproduct stream.

In some embodiments, the treated stream includes about 0 ppbw to less than about 900 ppbw of phosphorus, calculated on an elemental basis. In some embodiments, the treated stream includes about 0 ppbw to less than about 500 ppbw of phosphorus, calculated on an elemental basis. In some embodiments, the treated stream includes less than about 95%, e.g., less than about 90%, of the phosphorus present in the pyrolysis oil before contacting the byproduct stream.

In some embodiments, the treated stream includes about 0 ppbw to less than about 3,000 ppbw of chlorine, calculated on an elemental basis.

In some embodiments, the treated stream includes about 0 ppbw to less than about 2,000 ppbw of fluorine, calculated on an elemental basis.

In some embodiments, at least a portion of the treated stream including pyrolysis oil is directed to a hydrocracking reactor. For example, in some embodiments, a hydrocracking feed stream including at least a portion of the treated stream including pyrolysis oil and hydrogen are contacted with a hydrocracking catalyst to form a hydrocracked stream including ethane. Suitable methods and systems for hydrocracking are known in the art. For example, the hydrocracking feed stream can be contacted with a noble metal hydrocracking catalyst (e.g., zeolite-supported palladium (Pd)) at about 290° C. to about 450° C., and about 800 psig to about 30,000 psig. In some embodiments, the hydrocracking reactor includes a fixed-bed reactor.

In some embodiments, at least a portion of the hydrocracked stream including ethane is directed to a steam cracking reactor. For example, in some embodiments, a steam cracking feed stream including water and at least a portion of the hydrocracked stream including ethane is heated to form a cracked stream including ethylene. Suitable methods and system for hydrocracking are known in the art.

The pyrolysis oils described herein can be formed by contacting a plastic with a pyrolysis catalyst. For example, in some embodiments, the pyrolysis oil is formed by contacting a mixed polyolefin, a polyethylene, a polypropylene, a polystyrene, a polyvinyl chloride, a polyethylene terephthalate, or any combination thereof with a pyrolysis catalyst. Suitable methods and systems for pyrolysis are known in the art.

The treated stream from the liquid-liquid extractor can be washed with water, for example, before introduction to a hydrocracking reactor. In some examples, washing the treated stream with water in a second liquid-liquid extractor can remove unreacted acetic acid from the treated stream. In some embodiments, the water contacted in the second liquid-liquid extractor includes water separated from the byproduct stream (e.g., by distillation or liquid-to-liquid extraction, as described herein).

The second liquid-liquid extractor can be configured to contact the treated stream including acetic acid and pyrolysis oil with water to form a mixture including the pyrolysis oil, acetic acid, and water, and then separate the mixture to form a washed stream including the pyrolysis oil and a second waste stream including water and at least a portion of the acetic acid from the treated stream. The second liquid-liquid extractor can be any liquid-liquid extractor described herein.

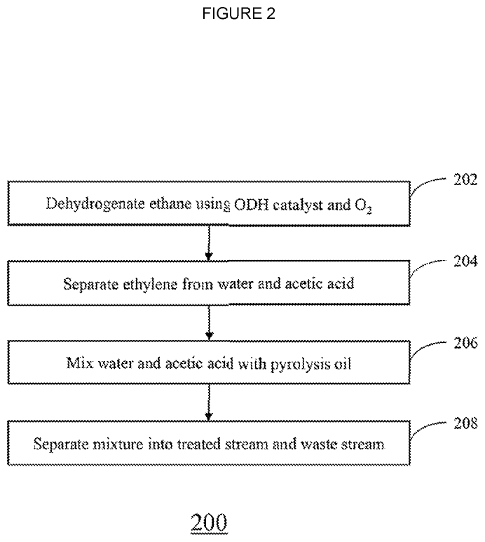

FIG. 2 is a process flow diagram of a method 200 for treating hydrocarbons. The method starts at block 202 with the contacting of an oxidative dehydrogenation feed stream including ethane and oxidant stream including oxygen with an oxidative dehydrogenation catalyst to form a dehydrogenated stream including ethylene and water. At block 204, at least a portion of the dehydrogenated stream is separated to form a gaseous product stream including ethylene and a byproduct stream including water and acetic acid. At block 206, a pyrolysis oil and at least a portion of the byproduct stream including water and acetic acid are contacted to form a mixture including the pyrolysis oil, water, and acetic acid. At block 208, the mixture is separated to form a treated stream including a portion of the pyrolysis oil and a waste stream including water and at least a portion of the contaminant or a derivative thereof.

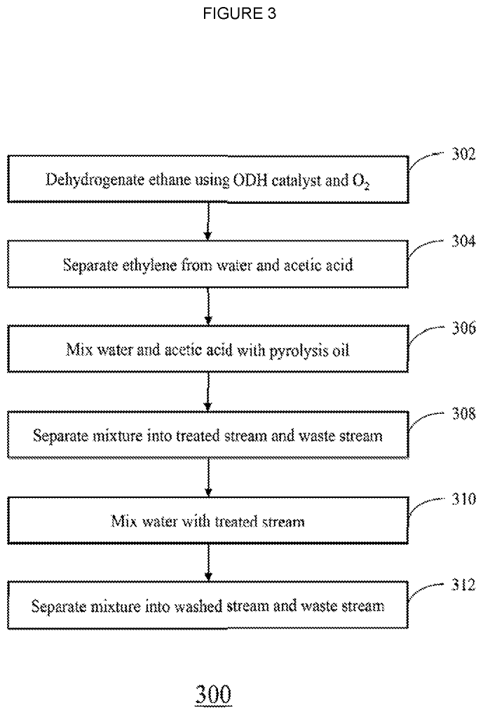

FIG. 3 is a process flow diagram of a method 300 for treating hydrocarbons. The method starts at block 302 with the contacting of an oxidative dehydrogenation feed stream including ethane and oxidant stream including oxygen with an oxidative dehydrogenation catalyst to form a dehydrogenated stream including ethylene and water. At block 304, at least a portion of the dehydrogenated stream is separated to form a gaseous product stream including ethylene and a byproduct stream including water and acetic acid. At block 306, a pyrolysis oil and at least a portion of the byproduct stream including water and acetic acid are contacted to form a mixture including the pyrolysis oil, water, and acetic acid. At block 308, the mixture is separated to form a treated stream including a portion of the pyrolysis oil and a portion of the acetic acid, and a waste stream including water and at least a portion of the contaminant or a derivative thereof. At block 310, water and at least a portion of the treated stream including pyrolysis oil and acetic acid are contacted to form a mixture including the pyrolysis oil, water, and acetic acid. At block 312, the mixture is separated to form a washed stream including at least a portion of the pyrolysis oil and a second waste stream including water and at least a portion of the acetic acid from the treated stream.

### Definitions

The terms “a,” “an,” and “the” are used herein to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed in this disclosure, and not otherwise defined, is for the purpose of description only and not of limitation. Any use of section headings is intended to aid reading of the document and is not to be interpreted as limiting; information that is relevant to a section heading may occur within or outside of that particular section.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (for example, 1%, 2%, 3%, and 4%) and the sub-ranges (for example, 0.1% to 0.5%, 1.1% to 2.2%, 3.3% to 4.4%) within the indicated range. The statement “about X to Y” has the same meaning as “about X to about Y”, unless indicated otherwise. Likewise, the statement “about X, Y, or about Z” has the same meaning as “about X, about Y, or about Z”, unless indicated otherwise.

As used herein, the term “about” can allow for a degree of variability in a value or range, for example, within 10%, within 5%, or within 1% of a stated value or of a stated limit of a range.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### EXAMPLES

The invention is further described in the following examples, which do not limit the scope of the invention described in the claims.

### Example 1. Pyrolysis Oil Extraction

Pyrolysis oil was prepared by pyrolyzing polypropylene at 460-470° C. The oil included 10-40 wt % paraffin/isoparaffin, 5-9 wt % olefin, 3-19 wt % naphthene, 9-10 wt % aromatics, and 28-47 wt % C₁₅₊ hydrocarbons. The pyrolysis oil was doped with contaminants according to Table 1, to reflect the composition of a typical pyrolysis oil derived from recycled plastic. An aqueous 21 wt % solution of acetic acid was added to the pyrolysis oil in a weight ratio of about 1:1. The mixture was vigorously mixed by hand, shaking for about 10 seconds, and then allowed to separate into an organic phase and an aqueous phase. Samples of the original pyrolysis oil and the separated organic phase were analyzed by inductively coupled plasma mass spectrometry (ICP-MS). Results are shown in Table 1, below.

TABLE 1
ICP-MS Analysis

| Element | Unit | Original | Treated | % Removed |
|---|---|---|---|---|
| Na | ppbw | 10,620 | 264.9 | 98% |
| P | ppbw | 961.7 | 829.1 | 14% |
| S | ppbw | 17,620 | 9,486 | 46% |
| K | ppbw | 41,820 | 33,590 | 20% |
| Cr | ppbw | 9.037 | 7.375 | 18% |
| Fe | ppbw | 226.8 | 129.5 | 43% |
| Ni | ppbw | 22.61 | 15.44 | 32% |
| Cu | ppbw | 34.42 | 1.847 | 95% |

As shown in Table 1, extraction with aqueous acetic acid reduced the amount of metal and heteroatom impurities present in the pyrolysis oil.

Other implementations are also within the scope of the following claims.

1. A method of treating hydrocarbons, the method comprising:
contacting an oxidative dehydrogenation feed stream comprising ethane and oxygen with an oxidative dehydrogenation catalyst to form a dehydrogenated stream comprising ethylene, water, and acetic acid;
separating at least a portion of the dehydrogenated stream to form a gaseous product stream comprising ethylene and a byproduct stream comprising water and acetic acid;
contacting a pyrolysis oil comprising a contaminant with at least a portion of the byproduct stream comprising water and acetic acid to form a mixture comprising the pyrolysis oil, water, and acetic acid; and
separating the mixture to form a treated stream comprising a portion of the pyrolysis oil and a waste stream comprising water and at least a portion of the contaminant or a derivative thereof.

2. The method of claim 1, wherein the dehydrogenation feed stream further comprises a diluent.

3. The method of claim 2, wherein the diluent comprises steam, carbon dioxide, or both.

4. The method of claim 1, wherein the dehydrogenated stream further comprises ethane, carbon dioxide, carbon monoxide, or any combination thereof.

5. The method of claim 1, wherein the product stream further comprises ethane, carbon dioxide, carbon monoxide, or any combination thereof.

6. The method of claim 1, wherein the contaminant comprises arsenic, calcium, iron, lead, aluminum, mercury, zinc, potassium, silica, sodium, copper, nickel, vanadium, sulfur, phosphorus, chlorine, a fluoride, or any combination thereof.

7. The method of claim 1, wherein the derivative of the contaminant comprises an acetate salt.

8. The method of claim 1, wherein the treated stream comprises about 0 ppbw to less than about 5 ppbw arsenic, calculated on an elemental basis.

9. The method of claim 1, wherein the treated stream comprises about 0 ppbw to less than about 500 ppbw of calcium, calculated on an elemental basis.

10. The method of claim 1, wherein the treated stream comprises about 0 ppbw to less than about 8 ppbw of chromium, calculated on an elemental basis.

11. The method of claim 1, wherein the treated stream comprises about 0 ppbw to less than about 150 ppbw of iron, calculated on an elemental basis.

12. The method of claim 1, wherein the treated stream comprises about 0 ppbw to less than about 50 ppbw of lead, calculated on an elemental basis.

13. The method of claim 1, wherein the treated stream comprises about 0 ppbw to less than about 1 ppbw of aluminum, calculated on an elemental basis.

14. The method of claim 1, wherein the treated stream comprises about 0 ppbw to less than about 5 ppbw of mercury, calculated on an elemental basis.

15. The method of claim 1, wherein the treated stream comprises about 0 ppbw to less than about 1 ppbw of zinc, calculated on an elemental basis.

16. The method of claim 1, wherein the treated stream comprises about 0 ppbw to less than about 35,000 ppbw of potassium, calculated on an elemental basis.

17. The method of claim 1, wherein the treated stream comprises about 0 ppbw to less than about 1,000 ppbw of silicon, calculated on an elemental basis.

18. The method of claim 1, wherein the treated stream comprises about 0 ppbw to less than about 300 ppbw of sodium, calculated on an elemental basis.

19. The method of claim 1, wherein the treated stream comprises about 0 ppbw to less than about 50,000 ppbw of copper, calculated on an elemental basis.

20. The method of claim 1, wherein the treated stream comprises about 0 ppbw to less than about 100,000 ppbw of nickel, calculated on an elemental basis.

21. The method of claim 1, wherein the treated stream comprises about 0 ppbw to less than about 50 ppbw of vanadium, calculated on an elemental basis.

22. The method of claim 1, wherein the treated stream comprises about 0 ppbw to less than about 500,000 ppbw of sulfur, calculated on an elemental basis.

23. The method of claim 1, wherein the treated stream comprises about 0 ppbw to less than about 900 ppbw of phosphorus, calculated on an elemental basis.

24. The method of claim 1, wherein the treated stream comprises about 0 ppbw to less than about 3,000 ppbw of chlorine, calculated on an elemental basis.

25. The method of claim 1, wherein the treated stream comprises about 0 ppbw to less than about 2,000 ppbw of fluorine, calculated on an elemental basis.

26. The method of claim 1, wherein the byproduct stream comprises about 0.1 wt % to about 30 wt % of acetic acid.

27. The method of claim 1, wherein the byproduct stream comprises about 1 wt % to about 30 wt % of acetic acid.

28. The method of claim 1, wherein the mixture comprises an emulsion of the pyrolysis oil and the byproduct stream.

29. The method of claim 1, wherein a weight ratio of the pyrolysis oil to the byproduct stream present in the mixture is about 5:1 to about 1:5.

30. The method of claim 1, wherein a weight ratio of the pyrolysis oil to the byproduct stream present in the mixture is about 2:1 to about 1:2.

31. The method of claim 1, wherein the waste stream further comprises acetic acid.

32. The method of claim 1, wherein the treated stream further comprises acetic acid.

33. The method of claim 32, further comprising
contacting at least a portion of the treated stream comprising pyrolysis oil and acetic acid with water to form a mixture comprising the pyrolysis oil, water, and acetic acid; and
separating the mixture to form a washed stream comprising at least a portion of the pyrolysis oil and a second waste stream comprising water and at least a portion of the acetic acid from the treated stream.

34. The method of claim 1, further comprising pyrolyzing a plastic to form at least a portion of the pyrolysis oil comprising the contaminant.

35. The method of claim 1, further comprising contacting a hydrocracking feed stream comprising at least a portion of the treated stream comprising pyrolysis oil and hydrogen with a hydrocracking catalyst to form a hydrocracked stream comprising ethane.

36. The method of claim 35, further comprising heating a steam cracking feed stream comprising water and at least a portion of the hydrocracked stream comprising ethane to form a cracked stream comprising ethylene.

37. A system for treating hydrocarbons, the system comprising:
an oxidative dehydrogenation reactor configured to contact an oxidative dehydrogenation feed stream comprising ethane and oxygen with an oxidative dehydrogenation catalyst to form a dehydrogenated stream comprising ethylene, water, and acetic acid;
a liquid-gas separator configured to separate at least a portion of the dehydrogenated stream to form a gaseous product stream comprising ethylene and a byproduct stream comprising water and acetic acid; and
a liquid-liquid extractor configured to
contact a pyrolysis oil comprising a contaminant with at least a portion of the byproduct stream comprising water and acetic acid to form a mixture comprising the pyrolysis oil, water, and acetic acid, and
separate the mixture to form a treated stream comprising a portion of the pyrolysis oil and a waste stream comprising water and at least a portion of the contaminant or a derivative thereof.

38. The system of claim 37, wherein the liquid-liquid extractor comprises an extraction column configured to form the mixture and separate the mixture.

39. The system of claim 37, wherein the liquid-liquid extractor comprises a static mixer configured to form the mixture and a liquid-liquid separator configured to separate the mixture.

40. The system of claim 37, further comprising a second liquid-liquid extractor configured to
contact at least a portion of the treated stream comprising pyrolysis oil and acetic acid to form a mixture comprising the pyrolysis oil, water, and acetic acid, and
separate the mixture to form a washed stream comprising at least a portion of the pyrolysis oil and a second waste stream comprising water and at least a portion of the acetic acid from the treated stream.

41. The system of claim 37, further comprising a hydrocracking reactor configured to contact a hydrocracking feed stream comprising at least a portion of the treated stream comprising pyrolysis oil and hydrogen with a hydrocracking catalyst to form a hydrocracked stream comprising ethane.

42. The system of claim 41, further comprising a hydrocracking reactor configured to heat a steam cracking feed stream comprising water and at least a portion of the hydrocracked stream comprising ethane to form a cracked stream comprising ethylene.
